(19) United States
(12) Patent Application Publication
(10) Pub. No.: US 2009/0264543 A1
(43) Pub. Date: Oct. 22, 2009

(54) Integrated Process for the Co-Production of Methanol and Demethyl Ether From Syngas Containing Nitrogen

(75) Inventors: Hengyong Xu, Liaoning (CN); Qingjie Ge, Liaoning (CN); Chunhua Tang, Liaoning (CN); Shoufu Hou, Liaoning (CN); Wenzhao Li, Liaoning (CN)

Correspondence Address: NIXON & VANDERHYE, PC, 901 NORTH GLEBE ROAD, 11TH FLOOR, ARLINGTON, VA, 22203, US

(73) Assignees: BP, P.L.C., London (GB); Dalian Institute of Chemical Physics Chinese Academy of Sciences, Liaoning (CN)

(21) Appl. No.: 11/989,327

(22) PCT Filed: Aug. 1, 2005
(86) PCT No.: PCT/CN2005/001173

§ 371 Date: Apr. 29, 2008

(51) Int. Cl. C07C 27/06

(52) U.S. Cl. 518/728

(57) ABSTRACT

The present invention relates to a novel integrated process for the co-production of methanol and dimethyl ether (DME) from syngas containing nitrogen, which is based on a two-stage reaction. In the first stage, most of the syngas is converted into methanol by using one reactor or two tandem reactors or multistage series reactors. In the second stage, the small amount of remaining syngas is further diluted by N2 and is converted to DME in the following reactor. Thus, the catalyst sintering is avoided due to the alleviated heat transfer limitations. An overall CO single pass conversion as high as ^{˜}90% is obtained, which is maintained during 2000 h's of continuous operation. This invention provides a novel, economic and easy to operate process to convert syngas to methanol/DME in single pass.

### FIELD OF THE INVENTION

The present invention relates to a novel process for the co-production of methanol and dimethyl ether (DME) from syngas containing nitrogen (N₂). The present invention further relates to a novel process for the co-production of methanol and dimethyl ether (DME) from syngas containing N₂, said process comprising two stages characterized in that the syngas containing N₂ is converted to methanol in the first stage and subsequently the unreacted syngas containing N₂ from stage 1 is converted to DME in the second stage.

By using this said integrated process consisting of a two-stage reaction, an overall CO single pass conversion as high as 90% was unexpectedly obtained.

### BACKGROUND OF THE INVENTION

Methanol is a major chemical raw material. Present global consumption is about 27 million tons per year. Major uses of methanol include the production of acetic acid, formaldehyde, and methyl-t-butylether. The latter, an oxygenate additive to gasoline, accounts for about a third of all use. Worldwide demand for methanol is expected to increase as much as five fold over the next decade as potential new applications become commercialized. Such applications include the conversion of methanol to gasoline, the conversion of methanol to light olefins, the use of methanol for power generation, and the use of methanol for fuel-cell powered automobiles.

In general, methanol synthesis is based on the equilibrium reactions of synthesis gas, namely reactions (1) and (2):

CO+2H₂→CH₃OH (1)

CO₂+3H₂→CH₃OH+H₂O (2)

The forward reactions (1) and (2) are exothermic, that is, they result in the formation of net heat. Also, the forward reactions (1) and (2) generate a lower volume of MeOH (gas) than the volume of feed (gas) used to form the methanol. Therefore, to maximize methanol yields, i.e., force reactions (1) and (2) to the right, the process requires low temperatures and high pressures for high conversion. Still, a typical methanol reactor will convert only about 20% to 60% of the synthesis gas fed to the reactor in a single pass through. To obtain higher conversions the unreacted synthesis gas is separated from the product methanol and recycled back to the reactor or directed to a second reactor to produce additional methanol. The conventional methanol synthesis catalysts are Cu-based catalysts which is often used at 210˜250° C., 2.0˜5.0 MPa.

DME (dimethyl ether) has received much attention as an alternative diesel fuel due to its low NOx emission and near zero smoke. Conventionally, DME has been produced by methanol dehydration which has a small production scale and high production cost, hence a more economic approach is to synthesize DME in a single step with hybrid catalysts (methanol synthesis catalyst and solid acid catalyst). The direct synthesis process mainly includes the following reactions:

2CO+4H₂=CH₃OCH₃+H₂O ΔH=−218.4 kJ/mol (523 K) (2)

3CO+3H₂=CH₃OCH₃+CO₂ ΔH=−257.9 kJ/mol (523 K) (3)

The above reactions show that the synthesis process of DME generates much more net heat than that of methanol.

At present, a lot of papers have reported on the study of syngas to DME in catalyst preparation, reactor, and process. Topsoe corporation developed a process of syngas to DME which aimed at natural gas resources, the autothermal reforming (ATR) syngas production process and fixed-bed reactor DME production process were used in this integrated process, the DME production operation conditions are: 4.2 MPa and 240˜290° C., the syngas feedstock need recycled, 50 kg-DME/d DME pilot test was finished in 1995. Air Product corporation and NKK corporation have developed the syngas to DME process which based on coal-bed methane resources, respectively. Air Product corporation finished a 4 ton/day DME production experiment in 1991 with LPDME (Liquid phase DME) process; The ATR process and slurry-bed reactor process were used in NKK DME synthesis process, and finished a project of 5 ton-DME/day pilot plant in 2001. As DME is used as fuel, decreasing the DME production cost is still the main goal of syngas to DME process. The cost of feedstock directly affects the DME product; therefore, developing a cheap syngas production process and an integrated process from methane to DME is the research trend for DME production. Dimethyl ether is mainly used in aerosol propellant at present. It is also widely recognized as a potential substitute of LPG (Liquified Petroleum Gas) and diesel. In addition, DME could also be used as the feedstock of light alkenes.

It is known that the CO conversion is low in the methanol synthesis due to the thermodynamic equilibrium constraint compared to that in the DME synthesis which can reach more than 90% CO in a single pass conversion.

In order to prevent the sintering of the catalysts in fixed-bed reactors due to the strong exothermic reaction of DME synthesis, the CO conversion has to be kept low and the unreacted feedstock recycled, which results in a larger consumption of compression energy.

Although slurry bed reactors are more efficient in terms of the heat-exchange of catalysts and isothermal operation can be achieved due to the bigger thermal capacity and good diathermancy of the liquid medium (e.g. paraffin); the extra mass transfer resistance for the reactant gas to reach the catalyst surface will lower the CO conversion. In addition to this, synthesis gas is mainly produced by steam reforming of natural gas in industry at present by the following reaction:

CH₄+H₂O=CO+3H₂ ΔH=206 kJ/mol (4)

This is a high energy consumption process, due to the strongly endothermic reaction and the product is a hydrogen-rich syngas and has a H₂/CO ratio of higher than 3 from industrial process, which is not stoichiometrically correct for the direct production of downstream methanol and DME.

On the other hand, catalytic partial oxidation of methane to synthesis gas (POM) has attracted a lot of attention since the 1990's, due to its mild exothermic reaction.

CH₄+1/2O₂=CO+2H₂ ΔH=−36 kJ/mol (5)

The synthesis gas obtained has a ratio of H₂/CO=2/1, which is the right stoichiometry for the synthesis of methanol and DME. However, the POM process requires pure oxygen, which increases the capital investment dramatically for air separation equipment and oxygen production.

By using air or oxygen-rich air instead of pure oxygen to carry out the POM reaction (air-POM) and simultaneously combining the POM process with steam reforming and/or CO₂ reforming, syngas can not only be produced economically, but also the reaction heat produced can be utilized more effectively by combining the exothermic POM and endothermic steam reforming and/or CO₂ reforming. It also has the added advantage in that the syngas has the right H₂/CO ratio for the production of methanol and DME.

Using air or oxygen-rich air instead of pure oxygen in the POM process produces syngas containing nitrogen. Due to the low single pass conversion of CO in the synthesis of methanol, the feed gas has to be recycled, which increases capital investment and compression energy. Although a per-pass conversion of CO of up to 90% can be achieved for the synthesis of DME from syngas containing N₂, the feedstock recycle is still necessary, because the CO conversion has to be kept low in order to alleviate the catalysts sintering due to the heat released in this highly exothermic reaction. Hence, the key issue for DME synthesis is how to improve the heat transfer efficiency for the said highly exothermic reaction whilst maintaining a high CO single pass conversion.

### SUMMARY OF THE INVENTION

The objective of the present invention is to provide an integrated process for the co-production of methanol and DME from cheap syngas containing N₂, which not only avoids the heat transfer limitations of the highly exothermic reaction but also maintains a high CO single pass conversion.

Thus, the present invention consists of a process for the co-production of methanol and dimethyl ether (DME) from syngas containing N₂, the said process consists of two stages characterized in that the syngas containing N₂ is converted to methanol in the first stage and the unreacted syngas containing N₂ from stage 1 is then converted into DME during the second stage.

According to a preferred embodiment of the present invention, most of the syngas is converted to methanol in the first stage; said first stage is performed preferably in either one reactor, two tandem reactors or multistage series reactors. The unconverted syngas from stage 1 is then converted to DME in the second stage of the process in a different reactor.

An overall CO single pass conversion as high as 90% can unexpectedly be obtained by using this process according to the present invention.

### BRIEF DESCRIPTION OF THE DRAWINGS

The novel integrated process according to the present invention is schematically represented in the figures.

FIG. 1 represents a schematic diagram of an integrated process embodiment according to the present invention for the co-production of methanol and DME from syngas containing N₂.

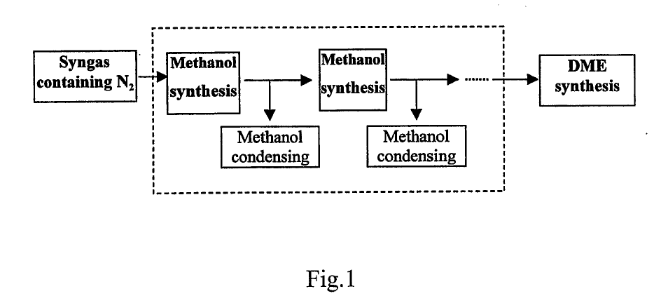

FIG. 2 is a table giving the CO conversion as a function of time stream in an embodiment of the integrated process of the present invention for the co-production of methanol and DME from syngas containing N₂.

### DETAILED DESCRIPTION OF THE INVENTION

By using this novel process, the major problem of the catalyst sintering is alleviated. This is because most of the syngas has been converted into methanol during the first stage of the process, and the remaining syngas used in the second stage i.e. for the DME synthesis, is further diluted by N₂ which will not result in a severe heat transfer limitation.

A high overall CO single pass conversion (˜90%) indicates that it is no longer necessary to recycle the feed gas, which hence saves the capital costs for the syngas recycle compressor and compression energy. Besides, the negative impact of N₂ can be ignored.

In principle, any kind of catalyst for the conversion of syngas to methanol and/or DME can be used in the integrated process of the present invention. For the well known catalysts used for the conversion of syngas to methanol and/or DME, the reaction conditions are: 190 to 290° C., 3.0 to 8.0 MPa, 200 to 2000 h⁻¹. Temperatures and pressures outside of the stated limits are not excluded, however they do not fall under the preferred embodiments of the present invention.

This invention will be further described through the following examples.

Unless otherwise indicated, the percentage (%) in the present invention means % by mole.

### EXAMPLES

### Example 1

The methanol synthesis was carried out in two tandem reactors and then the DME was synthesized in the following reactor. 1.5 g of Cu/ZnO/Al₂O₃ catalyst, prepared by coprecipitation method with 2:1:0.2 of Cu:Zn:Al atom ratio, was loaded into each of the tandem reactors, and 3.0 g of Cu/ZnO/Al₂O₃+HZSM-5 catalysts, prepared by coprecipitation-sedimentation method with 3:1 mass ratio of Cu/ZnO/Al₂O₃:HZSM-5 (derived of Nankai University), were loaded into DME synthesis reactor. The catalysts were reduced at 210° C. for 4 h after increasing the temperature from room temperature to 210° C. at a heating rate of 1° C./min in 5%H₂—Ar. The feeding gas was then switched to syngas containing N₂ (H₂/CO=2, balanced with 25% N₂) and the methanol/DME synthesis reaction was carried out under 4.0 MPa, 1000 h⁻¹, and 205° C. (methanol synthesis reactors), 210° C. (DME synthesis reactor). The experimental results show that 55% CO conversion is obtained for methanol synthesis in tandem reactors and an overall CO single pass conversion of 90% is achieved for methanol/DME synthesis according to the present integrated process.

### Example 2

The reaction conditions were the same as those in Example 1 except that the reaction pressure used was 5.0 MPa. and the feeding gas comprised 0.60% CH₄, 7.13% CO₂, 20.02% CO, 41.51% H₂, and 30.73% N₂; which are all products of the reaction between CH₄—H₂O-Air-CO₂ (molar ratio: 1/0.812.4/0.4) at 850° C., 0.8 MPa. The experimental results show that 54% CO conversion is obtained for methanol synthesis and an overall CO single pass conversion of 90% is achieved for the methanol/DME synthesis according to the present integrated process. When the DME synthesis was carried out at 215° C., the overall CO single pass conversion was shown to increase to 94% for the synthesis of methanol/DME.

### Example 3

The reaction conditions were the same as those in Example 1 except for the following conditions: 5.0 MPa, and a catalyst comprising Cu/ZnO/ZrO₂+HUSY, prepared by coprecipitation-sedimentation method with 2.3:1:0.2 of Cu:Zn:Zr atom ratio and 3:1 mass ratio of Cu/ZnO/ZrO₂:HUSY (derived of Nankai University), was used for the DME synthesis, and the feed gas comprised 0.60% CH₄, 7.13% CO₂, 20.02% CO, 41.51% H₂, and 30.73% N₂, all derived from the reaction between CH₄—H₂O-Air-CO₂ (molar ratio of 1/0.8/2.4/0.4) at 850° C., 0.8 MPa. 55% CO conversion was obtained for the methanol synthesis and an overall CO single pass conversion of 92% for the synthesis of methanol/DME could be obtained according to the present integrated process.

### Example 4

The reaction conditions are the same as those in Example 1 except for the following conditions: 5.0 MPa, and a catalyst comprising Cu/ZnO/ZrO₂+Al₂O₃+HZSM-5, prepared by coprecipitation-sedimentation method with 2.3:1:0.2 of Cu:Zn:Zr atom ratio and 3:1 mass ratio of Cu/ZnO/ZrO₂:(Al₂O₃+HZSM-5) (20% wt of Al₂O₃/(Al₂O₃+HZSM-5), Al₂O₃ is bought from Shandong Alumina Corporation, and HZSM-5 is from Nankai University), was used for the DME synthesis, and the feed gas comprised 0.60% CH4, 7.13% CO₂, 20.02% CO, 41.51% H₂, and 30.73% N₂, all derived from the reaction between CH₄—H₂O-Air-CO₂ (molar ratio of CH₄/H₂O/Air/CO₂=1/0.8/2.4/0.4) at 850° C., 0.8 MPa. The experimental results show that CO conversion of 54% was obtained for the methanol synthesis and an overall CO single pass conversion of 89% was obtained for the methanol/DME synthesis according to the present integrated process.

### Example 5

The reaction conditions were the same as those in Example 1 except for the feed gas comprised 0.86% CH₄, 9.11% CO₂, 22.8% CO, 44.5% H₂, and 22.8% N₂; all products of the reaction between CH₄—H₂O-Air(oxygen-rich)-CO₂ (molar ratio: 1/0.8/1.47/0.4) at 850° C., 0.8 MPa. The experimental results show that 54% CO conversion was obtained for the synthesis of methanol and an overall CO single pass conversion of 90% was obtained for the methanol/DME synthesis according to the present integrated process.

### Example 6

The reaction conditions are the same as those used in Example 1 except that the feed gas comprised 1.08% CH₄, 5.84% CO₂, 17.6% CO, 51.7% H₂, and 23.8% N₂; all products of the reaction between CH₄—H₂O-Air(oxygen-rich) (molar ratio: 1/0.8/1.47) at 850° C., 0.8 MPa. The experimental results show that a 56% CO conversion was achieved for the methanol synthesis and an overall CO single pass conversion of 94% was obtained for the methanol/DME synthesis according to the integrated process.

### Example 7

The reaction conditions are the same as those used in Example 1 except that the feed gas comprised of 0.66% CH₄, 4.69% CO₂, 14.5% CO, 42.4% H₂, and 37.7% N₂; all products from the reaction between CH₄—H₂O-Air (molar ratio 1/0.8/2.4) at 850° C., 0.8 Ma. The experimental results show that a 56% CO conversion was achieved for the methanol synthesis and an overall CO single pass conversion of 94% was obtained for the methanol/DME synthesis according to the integrated process.

### Example 8

One reactor was used for the methanol synthesis and a following reactor was used for the DME synthesis. 2 g of Cu/ZnO/Al₂O₃ catalyst (the composition is the same as that shown in example 1) was used for the methanol synthesis and 2 g of Cu/ZnO/Al₂O₃+HZSM-5 DME synthesis catalysts (the composition is the same as that shown in example 1) were loaded into each of the reactors respectively. The catalysts were then reduced at 210° C. for 4 h after they had been heated from room temperature to 210° C. at a heating rate of 1° C./min in 5%H₂—Ar. The feed gas was then switched to syngas containing N₂ and the methanol/DME synthesis reaction was performed at 215° C., 5.0 MPa, 1000 h⁻¹, with a feed gas comprising (0.60% CH₄, 7.13% CO₂, 20.02% CO, 41.51% H₂, and 30.73% N₂); all products of the reaction between CH₄—H₂O-Air-CO₂ (molar ratio 1/0.8/2.4/0.4) at 850° C., 0.8 MPa. The experimental results show that a 50% CO conversion for the methanol synthesis was obtained and an overall CO single pass conversion of 90% was obtained for the methanol/DME synthesis according to the present integrated process.

### Example 9

The reaction conditions are the same as those used in Example 8 except that the feed gas comprised 0.66% CH₄, 4.69% CO₂, 14.5% CO, 42.4% H₂, and 37.7% N₂; all derived from the reaction between CH₄—H₂O-Air (molar ratio 1/0.8/2.4) at 850° C., 0.8 MPa. The experimental results show that 55% CO conversion was obtained for the methanol synthesis and an overall CO single pass conversion of 94% was obtained for the methanol/DME synthesis according to the present integrated process.

### Example 10

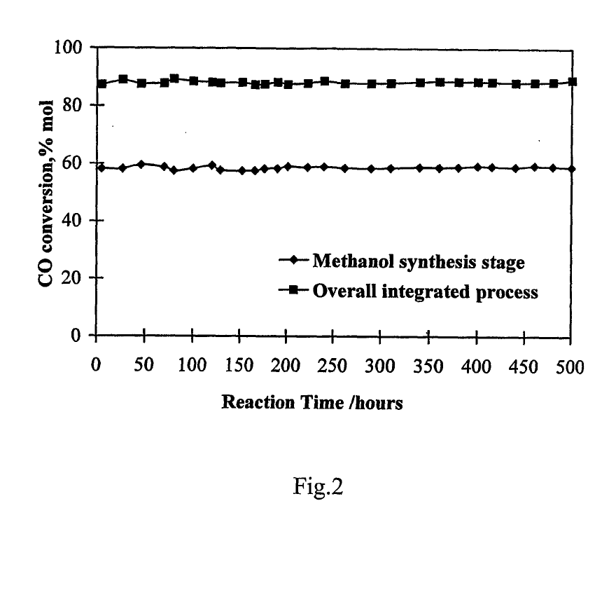

The methanol was synthesized in two tandem reactors and the DME was synthesized in a following reactor. 1.5 g of Cu/ZnO/Al₂O₃ catalyst (the composition is the same as that shown in example 1) was used for the methanol synthesis and was loaded into each of the tandem reactors, and 3.0 g of Cu/ZnO/Al₂O₃+HZSM-5+Al₂O₃, prepared by coprecipitation-sedimentation method with 2:1:0.2 of Cu:Zn:Al atom ratio and 3:1 mass ratio of Cu/ZnO/Al₂O₃:(Al₂O₃+HZSM-5) (20% wt of Al₂O₃/(Al₂O₃+HZSM-5), Al₂O₃ is bought from Shandong Alumina Corporation, and HZSM-5 is from Nankai University), catalysts were loaded into the DME synthesis reactor. The catalysts were then reduced at 210° C. for 4 h after they had been heated from room temperature to 210° C. at a heating rate of 1° C./min in 5%H₂-Ar. The feed gas was then switched to syngas containing N₂ for and the methanol/DME synthesis reaction was performed at 5.0 MPa, 1000 h⁻¹. The composition of the feed gas was 0.60% CH₄, 7.13% CO₂, 20.02% CO, 41.51% H₂, and 30.73% N₂, all derived from the reaction of CH₄—H₂O-Air-CO₂ (molar ratio 1/0.8/2.4/0.4) at 850° C., 0.8 MPa. The experimental results show that a 58% CO conversion for methanol synthesis and an overall CO single pass conversion of 88% for methanol/DME synthesis were obtained and kept constant in the integrated process during 500 h's continuous operation (See FIG. 2).

### Example 11

The methanol was synthesized in two tandem reactors and the DME was then synthesized in a following reactor. 1.5 g of Cu/ZnO/Al₂O₃ catalyst (the composition is the same as that shown in example 1) was used for the methanol synthesis and was loaded into each of the tandem reactors, and 3.0 g of Cu/ZnO/Al₂O₃+HZSM-5+Al₂O₃ catalysts(the composition is the same as that shown in example 10) were loaded into DME synthesis reactor. The catalysts were reduced at 210° C. for 4 h after they had been heated from room temperature to 210° C. at a heating rate of 1° C./min in 5% H₂-Ar. The feed gas was then switched to syngas containing N₂ for the methanol/DME synthesis reaction under 5.0 MPa, 1000 h⁻¹. The composition of the feed gas was 0.50% CH₄, 8.41% CO₂, 17.71% CO, 35.89% H₂, and 37.14% N₂, derived from the reaction of CH₄—H₂O-Air-CO₂ (molar ratio: 1/0.8/2.4/0.3) at 850° C., 0.8 MPa. The experimental results show that a 56% CO conversion for methanol synthesis and an overall single pass CO conversion of 86% for the methanol/DME synthesis were obtained and kept constant in the integrated process during 2000 h's of continuous operation.

1. Process for the co-production of methanol and dimethyl ether (DME) from syngas containing N₂, said process consisting of two stages characterized in that the syngas containing N₂ is converted to methanol in the first stage and the unreacted syngas containing N₂ from stage 1 is then converted to DME in the second stage.

2. Process according to claim 1 wherein most of the syngas is converted to methanol in the first stage, said first stage being performed in one reactor or two tandem reactors or multistage series reactors.

3. Process according to claim 1 wherein the unconverted syngas from stage 1 is converted to DME in the second stage in a different reactor.

4. Process according to claim 1 wherein the process reaction conditions for the co-production of methanol and DME are reaction temperatures of 190 to 290° C., pressure of 3.0 to 8.0 MPa, and syngas GHSV of 500-2000 h″¹.

5. Process according to claim 1 wherein the syngas containing N₂ is derived from the combined process of air catalytic partial oxidation and steam reforming of natural gas.

6. Process according to claim 1 wherein the syngas containing N₂ is derived from the combined process of oxygen-rich air catalytic partial oxidation and steam reforming of natural gas.

7. Process according to claim 1 wherein the syngas containing N₂ is derived from the combined process of air catalytic partial oxidation and CO₂ reforming of natural gas.

8. Process according to claim 7 wherein the syngas containing N₂ is derived from the combined process of oxygen-rich air catalytic partial oxidation and CO₂ reforming of natural gas.

9. Process according to claim 1 wherein the syngas containing N₂ is derived from the combined process of air catalytic partial oxidation, steam reforming and CO₂ reforming of natural gas.

10. Process according to claim 9 wherein the syngas containing N₂ is derived from the combined process of oxygen-rich air catalytic partial oxidation, steam reforming and CO₂ reforming of natural gas.

11. Process according to claim 1 wherein the molar percentage of N₂ in syngas is in the range of 10% to 50% and preferably 20% to 40% in the first stage.

12. Process according to claim 1 wherein the molar percentage of N₂ in syngas is: in the range of 18 to 67% and preferably 33 to 57% in the second stage.

13. Process according to claim 1 wherein 20% to 60% of the syngas is converted to methanol in the first stage.
